(19) United States
(12) Patent Application Publication
(10) Pub. No.: US 2005/0004001 A1
(43) Pub. Date: Jan. 6, 2005

(54) Formulation of antigen

(76) Inventors: Robert Harris, Solna (SE); John Robertson, Stockholm (SE); Anders Essen-Moller, Stockholm (SE)

Correspondence Address: Roger A. Gilcrest;Schottenstein Zoe & Dunn, 250 East Broad Street, P. O Box 165020, Columbus, OH, 43216-5020, US

(21) Appl. No.: 10/677,767

(22) Filed: Oct. 2, 2003

Related U.S. Application Data
(60) Provisional application No. 60/415,494, filed on Oct. 2, 2002.

(51) Int. Cl. A61K 38/28; A61K 38/17

(52) U.S. Cl. 514/2; 514/3

(57) ABSTRACT

The present invention regards methods and formulations for diagnosis, prevention and treatment of disease. More particularly, the present invention teaches methods and formulations for diagnosis, prevention and treatment with antigen in autoimmune disease, allergy, rejection of transplants and cancer. Examples illustrate how the methods and formulations of the invention may be used for diagnosis and amelioration of autoimmune diabetes in which the 65kd isotype of glutamic acid decarboxylase (GAD) is a major antigen.

### CROSS-REFERENCE TO RELATED APPLICATIONS

This application claims the benefit of U.S. Provisional Patent Application No. 60/415,494, filed on Oct. 2, 2002, which is incorporated herein by reference.

### BACKGROUND AND SUMMARY OF THE INVENTION

The present invention regards methods and formulations for diagnosis, prevention and treatment of disease. More particularly, the present invention teaches methods and formulations for diagnosis, prevention and treatment with antigen in autoimmune disease, allergy, rejection of transplants and cancer. Examples illustrate how the methods and formulations of the invention may be used for diagnosis and amelioration of autoimmune diabetes in which the 65kd isotype of glutamic acid decarboxylase (GAD) is a major antigen.

Lymphocytes play fundamental roles in disease defense and pathogenesis, and much effort is currently directed to defining both disease-promoting and disease-protective T cell responses in order to facilitate diagnosis and development of appropriate therapies. A variety of organ-specific inflammatory diseases have been dissected with respect to the relative roles of CD4+ and CD8+ cells. In the non-obese diabetic (NOD) mouse, a murine model for diabetes mellitus, transfer of both CD4+ and CD8+ T cell clones cause islet infiltration and destruction (Wong F. S., 1999). In mouse collagen-induced arthritis (CIA) reported data suggest that CD4+ and CD8+ T cells could independently promote the development of CIA (Tada, 1996). Although Myasthenia Gravis is thought to be an antibody-driven autoimmune disease affecting neuromuscular junctions, both CD4+ and CD8+ cells are reported to be important for development of experimental autoimmune myasthenia gravis (EAMG) (Zhang, 1996). When the autoimmune mechanisms in experimental autoimmune myocarditis (EAM) were dissected it was concluded that neither CD4+ nor CD8+ T cells were essential for disease (Penninger, 1993 #88). Therefore, in different experimental autoimmune models, CD4+ and CD8+ T cells may play different roles in pathogenesis.

It is known in the art that naive regulatory T cells develop into various subsets of T helper cells upon antigen stimulation depending not only on the antigen itself and/or the patient's genetic predisposition but also on the context in which the antigen is presented. For example, in the NOD mouse, stimulation with antigen in association with Complete Freunds Adjuvant induces a response, where proinflammatory Th1 cells dominate over those giving a humoral response to associated Th2 cells. In contrast, if the antigen is administered with Incomplete Freunds Adjuvant, the Th2 response will be enhanced. Some of the activated Th cells will in turn develop into longlived memory T cells committed to a specific immune response upon re-stimulation.

The prevailing cytokine profile and the activity of the immune system may vary from time to time within an individual; for example an ongoing virus infection may in many cases alert the cell-mediated arm of the immune defense system more than a bacterial infection. Therefore, encounter with an antigen administered as a part of antigen specific therapy may affect the immune system differently depending on its actual status at the time of administration.

It is an object of the present invention to present a method for selecting the route of administration and formulation for antigen-specific therapy—aiming at accomplishing a disorder appropriate immunomodulation.

It is a further object of the present invention to disclose non-toxic immunoneutral formulations capable of solubilizing hydrophilic as well, as hydrophobic protein antigens.

It is yet a further object of the present invention to disclose a formulation with the GAD antigen capable of ameliorating beta cell specific inflammation in the islets of Langerhans in man.

### DETAILED DESCRIPTION OF THE INVENTION

In order to diagnose, prevent or treat autoimmune disease, transplant rejection, or cancer it is of increasing interest to expose various antigens to naive and activated lymphocytes in a case appropriate context, such that the lymphocyte response results in production of cytokines that promote the overall immune response to the particular antigen in a desired way. In transplant rejection and organ specific autoimmune disease, such as for example type I diabetes, downregulation of inflammation in or around the specific organ is desired whereas upregulation of specific cytotoxic lymphocytes is desired in treatment of cancer.

When stimulating lymphocytes with antigen, it is of particular importance that, apart from endotoxin levels being low, the formulation itself is immunoneutral while being able to keep an antigen in solution. This is so that the formulation itself does not stimulate the immune system in a particular direction.

As the status of an individual's immune system may vary from time to time, exposure to an antigen may give a different response from the immune system on different occasions. For example, it can be speculated that as viral disease activates the cell-mediated arm of the immune system with a characteristic Th1 cytokine profile, administration of unadjuvanted endogenous antigen may give rise to autoimmune disease in a susceptible individual. Once a lymphocyte response to an antigen solubilized in an immunoneutral formulation is defined at a particular time, the route of administration and formulation for an antigen specific therapeutic can be decided upon according to the method of the present invention—aiming at accomplishing appropriate immunomodulation for a given disorder.

The formulations of the present invention disclose non-toxic immunoneutral formulations capable of solubilizing hydrophilic as well as hydrophobic protein antigens. In addition the present innovation presents a formulation of GAD capable of downregulating IFN-gamma production in individuals with GAD reactive T cells—indicating a downregulation of beta cell destruction in the islets of Langerhans, and of inducing a non-inflammatory response to the GAD antigen for prevention and therapy of diabetes.

### EXPERIMENT I

In human diabetes, a number of GAD specific T cell clones have been identified in the peripheral blood of type 1 diabetes patients (Roep ref). As a starting point for such cloning, and as an integral part of clinical immunological testing, it is critical to be able to re-stimulate GAD-specific T cells derived from peripheral blood in vitro. The use of GAD in assays for in vitro re-stimulatation of T cells to elicit cytokine production and cellular proliferation, is an important and problematic issue that has led to the establishment of an International T Cell Workshop whose primary aim is to establish sources of diabetes-relevant antigens and appropriate protocols for their use. GAD has been expressed as a recombinant protein in bacteria, insect cells and in yeast. The consensus from this Workshop is that recombinant Diamyd™ GAD (commercially available from Diamyd, Inc. of Raleigh, N.C.), produced by baculovirus expression in insect cells, is appropriate and useful for in vitro T cell assays (Roep, 1999 #262; Peakman, March 2002).

However, the GAD preparations are still not ideal, and the experience of the T Cell Workshop can be summarized as: i) GAD in buffer comprising reducing agents and detergents is toxic to T cells; and ii) dialysis of GAD reduces toxicity but increases risk of precipitation.

In order to improve the suitability of proteins such as GAD or MOG (an antigen associated with multiple sclerosis) for human T cell work, new formulations were devised and tested for efficacy in different T cell assays. The premise for this formulation was to 1) reduce the toxicity inherent in the buffer solutions; 2) without by itself stimulating or directing a response from the immune system; and 3) while allowing full solubility of the protein, which is usually lost on direct buffer exchange to PBS. The formulation was unexpectedly achieved through addition of Human Serum Albumin (HSA) during the process of buffer exchange to cell culture medium (RPMI).

Although not limited to the theory of operation of the invention, it is believed that the addition of an immunogenetically ‘neutral’ protein would allow preferential interaction of the protein as its buffer is replaced with cell culture medium, preventing the protein molecules interacting with each other and thus preventing precipitation.

Cell culture medium was selected for buffer exchange, because this is the assay medium used for in vitro immunological assays, Human serum albumin (HSA) was selected as an example of an immunologically neutral protein, as this is a major component of human blood proteins and also of complete cell culture used in vitro assays. It would thus not cause induction of any type of immune response itself per se, and would therefore not itself impact on the reactions or functions of the T cells in the assay.

The process of protein precipitation occurs due to protein molecules developing a higher charge affinity to each other than to the surrounding medium, leading to protein-protein aggregation that becomes apparent as an insoluble precipitate. This occurs for example during buffer exchange of GAD in Diamyd™ buffer (commercially available from Diamyd, Inc.) to PBS. The electrostatic charge of GAD changes as the detergent and reducing agent in the Diamyd buffer is replaced by PBS.

Whereas the approach did not work for the MOG antigen which precipitated when it in a pH3 buffer was dialysed against HSA, the GAD antigen stayed perfectly in solution during similar treatment.

Optimisation of the formulation of T cell GAD was based on the solubility and T cell stimulatory capacities of different formulations studied in vitro. The concentrations of GAD and of HSA were varied in two different experimental series:

Series 1. Solubility Testing

| SAMPLE | GAD conc | HSA conc | Ratio | |
|---|---|---|---|---|
| I.D. | a4lml | mg/ml | GAD:HAS | Dialysis buffer |
| A | 6.5 | 3.25 | 2:1 | RPMI |
| B | 6.5 | 6.5 | 1:1 | RPMI |
| C | 2 | 1 | 2:1 | RPMI |
| D | 1 | 1 | 1:1 | RPMI |
| E | 1 | 0.5 | 2:1 | RPMI |
| F | 1 | 0 | 1:0 | RPMI |
| Control | 2.5 | 1 | 2.5:1 | RPMI |

Series 2. T Cell Stimulation

| SAMPLE | GAD conc | Albumin conc | Ratio | |
|---|---|---|---|---|
| I.D. | mg/ml | mg/ml | GAD:HSA | Dialysis Buffer |
| 1 | 1 | l (HSA) | 1:1 | RPMI |
| 2 | 1 | 0 | 1:0 | RPMI |
| 3 | 0 | 1 (HSA) | 0:1 | RPMI |
| 4 | 1 | 0 | 1:0 | Diamyd Buffer |
| 5 | 1 | 20 (HSA) | 1:20 | RPMI |
| 6 | 1 | 0 | 1:0 | ddH2O |
| 7 | 0 | 20 (HSA) | 0:20 | RPMI |
| 8 | 1 | 1 (MSA) | 1:1 | RPMI |
| 9 | 0 | 1 (MSA) | 0:1 | RPMI |

Formulations were assessed for precipitation by visual inspection and were additionally assessed for structural integrity and immunogenicity by SDS PAGE and western blotting with both N- and C-terminal specific monoclonal antibodies. Different T cell assays endpoints were measured as follows:

| CELLULAR SOURCE | ASSAY |
|---|---|
| human PBMC | proliferation |
| human PBMC | IFN-γ cell awface FACS |
| humn T cell lines | proliferation |
| human T cell lines | proliferation |
| NOD mouse PBMC | IFN-γ ELISPOT |
| NOD mouse T cell lines | proliferation |
| human serum | antibody radio immunoassay |

The concentration of the GAD dialysed is important. The most efficient GAD concentration was 1 mg/ml, with no detectable precipitate. At high concentrations (i.e. 6.54 mg/ml) there is still appreciable precipitation after dialysis.

The ratio of HSA:GAD is important. The most efficient HSA:GAD ratio was 1:1, with which there was no detectable precipitate. At more than 1:1 (HSA:GAD) there is increased precipitation, irrespective of the concentration of GAD in the sample.

Dialysis of GAD with RPPM, with or without the inclusion of HSA, gave stimulation of T cells in proliferation, cytokine capture and ELISPOT analyses. Irrespective of whether human PMBC or defined T cell lines, or mouse PBMC or defined mouse T cell lines were used in these assays, the dialysed GAD preparations performed better than the original non-dialysed counterpart.

The solubility of GAD greatly affected its T cell stimulatory ability, the least soluble preparations stimulating less efficiently than their soluble counterparts.

Based on the superior stimulation of T cells, the final formulation of tcGAD was that GAD65 should be dialysed at a concentration of 1 mg/ml with 1 mg/ml HSA against RPMI for use in in vitro T cell assays.

### EXPERIMENT 2

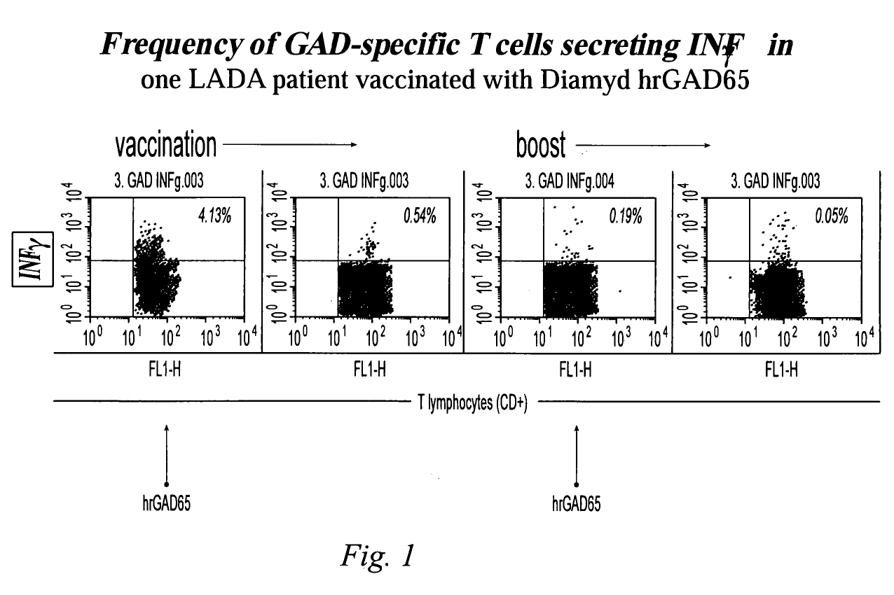

Glutarmic acid decarboxlase (GAD65) is an autoantigen proposed to be a major target of autoimmunity during initiation and maintenance of the inflammatory process leading to beta cell destruction, mid insulin dependency in man. T cells from type-2 diabetes patients with GAD antibodies were stimulated in vitro with GAD formulated in HSA before and after in vitro subcutaneous administration of GAD formulated in alum. As HSA is a common soluble protein in man, the GAD-HSA formulation was intended not to modify the status of the immune system. Stimulation with tetanus toxoid was used as control. IFN-gamma was measured with cytokine secretion assays. As is shown in FIG. 1 IFN-gamma secretion was dramatically reduced upon GAD-HSA in vivo stimulation after subcutaneous in vivo administration of GAD-alum. The effect was persistent over at least four weeks and after a subsequent subcutaneous boost.

1. A method to stimulate the immune system in vitro with an antigen in a formulation without interfering with the current preprogrammed response of the immune system to that antigen by means of administering the antigen in an otherwise immunoneutral formulation.

2. A method to modulate an undesired current status of the immune system in an individual by:
a) evaluating the current immune response to an antigen in vitro according to the method of claim 1.
b) deciding on a therapeutic formulation suitable for specific immunomodulation away from of the individual's current unwanted immune response.

3. A formulation that is immunoneutral to the immune system of an individual and capable of solubilizing hydrophobic antigens.

4. A formulation according to claim 3 where immunoneutral hydrophobic solvent is species specific Serum Albumin.

5. A formulation according to claim 4 including an antigen for stimulation of the immune system.

6. A formulation for antigen specific downregulation of inflammatory responses comprising at least two of the ingredients antigen and alum.

7. A formulation according to claim 5 or 6, where the antigen is of at least one from the group of ICA512 (IA2), ICA512B (IA2B), insulin, insulin B-chain, proinsulin, Hsp60, Hsp65, P277, ICA69, Glima38, GAD 65, GAD67, SOX13, Imogen 38, Sulfatide, MBP, MOG, Collagen II, 21-Ohase, TPO, allergens, transplant antigens, cancer antigens, or parts, peptides or altered peptide ligands thereof.

8. A kit for evaluation of lymphocyte reaction to antigen comprising a formulation according to claim 7.

9. A therapeutic comprising a formulation according to claim 7.

10. A therapeutic according to claim 9 where concentrations of antigen for in vitro stimulation typically is between 1-100 micrograms per ml.

11. A therapeutic according to claim 9 where concentrations of antigen for in vitro stimulation typically is between 5-40 micrograms per ml.

12. A therapeutic according to claim 9 where the subcutaneous administration of antigen for in vivo stimulation typically is between 0.1 micrograms per ml.

13. A therapeutic according to claim 9 where intravenous administration of antigen for in vivo stimulation typically is between 0.1-5 mgs/kg.
